(19) United States
(12) Patent Application Publication
(10) Pub. No.: US 2019/0021777 A1
(43) Pub. Date: Jan. 24, 2019

(54) Cryotherapy Device For The Treatment of Cervical Precancerous Lesions

(71) Applicants: Marton Varady, Baltimore, MD (US); Shuja T. Dawood, Santa Clara, CA (US); John-William Sidhom, Westfield, NJ (US); Enriquito Lu, Baltimore, MD (US)

(72) Inventors: Marton Varady, Baltimore, MD (US); Shuja T. Dawood, Santa Clara, CA (US); John-William Sidhom, Westfield, NJ (US); Enriquito Lu, Baltimore, MD (US)

(73) Assignee: Jhpiego Corporation, Baltimore, MD (US)

(21) Appl. No.: 15/655,092

(22) Filed: Jul. 20, 2017

(51) Int. Cl. A61B 18/02

(52) U.S. Cl. CPC A61B 18/0218; A61B 2018/00577; A61B 2018/0231

(57) ABSTRACT

A device for providing a patient with a cryotherapy process that is directed to the ablation of a lesion on the patient, includes: (a) a piping assembly adapted to create a spray of carbon dioxide snow, (b) a snow horn having a pressure relief means and a boundary wall, (c) a tubular applicator with ends between which extends an interior wall that has an interior diameter that increases in value between these ends, (d) an applicator tip with a metal portion and adapted to allow it to connect to the applicator's distal end, and (e) a push rod having a distal end with a base to which is attached an anchor.

### CROSS-REFERENCE TO RELATED APPLICATION

This is a continuation-in-part, patent application that claims the priority and benefits of U.S. patent application Ser. No. 13/898,962, filed May 21, 2013 by the present inventors and entitled “CRYOTHERAPY DEVICE AND METHOD FOR THE TREATMENT OF CERVICAL PRECANCEROUS LESIONS” and for which U.S. Pat. No. 9,717,546 will issue on Aug. 1, 2017. The teachings of this parent application are incorporated herein by reference to the extent that they do not conflict with the teaching herein.

### BACKGROUND OF THE INVENTION

### 1. Field of the Invention

The present invention relates generally to surgery devices and methods. More particularly, the present invention relates to a cryotherapy device and method to treat, destroy or ablate a patient's abnormal tissues or lesions, e.g., for the ablation of cervical, precancerous lesions to prevent cervical cancer.

### 2. Description of the Related Art

Cervical cancer is the third most common cancer in the world. Moreover, eighty percent of all cervical cancer cases occur in the developing world. With approximately 500,000 new cases each year, cervical cancer is responsible for over 250,000 deaths per year, making it the second leading cause of death in developing countries. Many of these deaths are women in their late 30s and early 40s, thus compromising the health and well-being of the surrounding family. Indeed, this is especially true for the children of these women, who often fall behind in their education and may be abandoned, without a mother.

While steps, such as annual PAP smears and other interventional methods, have been taken to eradicate cervical cancer in the developing world, cervical cancer still remains a large burden for these countries. Its prevalence in the developing world can in large part be attributed to a lack of appropriated technologies for screening and treatment.

A “single visit” approach has been developed and includes a screening by visual inspection with acetic acid and a point-of-diagnosis cryotherapy treatment. This approach for screening and treatment has provided a safe, acceptable, and feasible option in low-resource settings.

Cryotherapy includes freezing the abnormal cervical tissue with a coolant such as carbon dioxide, CO₂, and has been used for over forty years to treat cervical dysplasia. This process uses what is called a “double freeze procedure.” One freezes the cervix for three minutes and then allows it to thaw for five minutes, then it is frozen again for an additional three minutes. This procedure maximizes the amount of tissue that is frozen more so than just doing one extra-long freeze because the frozen tissue becomes more thermally conductive after the first freeze so the second freeze penetrates quicker and subsequently deeper into the affected tissue.

Cryotherapy is the leading method for the ablation of cervical precancerous lesions for the prevention of cervical cancer, especially in the developing world. Despite proven efficacy of cryotherapy as a mode of ablating precancerous lesions, the current state of the art used to facilitate cryotherapy is not sufficiently designed for widespread and reliable use in the developing world.

While this single visit approach or “screen and treat” program has been shown to be effective in prevention of cervical cancer, there still remains a hurdle to scaling up such a program for widespread impact. While cryotherapy using CO₂ has been shown to be safe and effective, even in the hands of low-level health care workers, the equipment is not necessarily suitable to be widely dispersed. Indeed, cryotherapy tools can be expensive, technically complex, lacking portability, and difficult to repair in the field.

Currently, the developing world utilizes cryoguns, see, for example, U.S. Pat. No. 4,377,168 which discloses expanding pressurized CO₂ (or N₂O in some instances when a country or program can afford the added expense of the N₂O) against a thermally conductive tip. The expansion of the CO2 in this closed system creates a temperature of approximately −50 to −70 degrees C. on the surface of the tip which is in contact with a lesion.

With carbonated beverage manufacturers being widely dispersed in the developing world, and often using carbon dioxide to produce their carbonated beverages, carbon dioxide manufacturers are also widely dispersed in the developing world. This has led to cryoablation being conducted with carbon dioxide as the primary source of coolant.

The current design of cryotherapy tools suffers from several flaws when utilized with carbon dioxide tanks, including: water vapor leaving the tank causes tip blockages, non-medical grade carbon dioxide may have particulates, which cause tip blockages, and a pressure drop may occur in the cylinder through the cooling process during extended use which results in warmer and less effective tip temperatures.

Additionally, the equipment was never originally designed for use within the extreme conditions of the developing world where there are high volumes of ablations along with environmental abuse of the product. First, the tips on the current cryotherapy tools are manufactured with gold or chrome plating, which not only makes the tip expensive, at approximately three hundred dollars per tip, but these tips also suffer from corrosion from the chlorine disinfection method—the widely available method for sterilization in the developing world.

Improper cleaning and storage of these tips also leads to clogs, requiring replacement. Additionally, the overall cost of the equipment is around thirteen hundred to two thousand dollars per device. Moreover, with the complexity of this equipment's engineering design, there is a lack of repair knowledge and backup parts when the equipment breaks down. Unfortunately, when the cryotherapy equipment malfunctions in the field, the device becomes unused and is rarely capable of being easily fixed.

Finally, the amount of carbon dioxide required per treatment limits the portability of the device. A tank containing 50 lbs of CO₂, weighs approximately 160 lbs total, and treats only about 10 to 15 patients. These large tanks are therefore difficult to transport to the rural areas where women need to be screened and treated, either in the context of health centers, mobile health vehicles or organized screen & treat camps. Therefore, these characteristics of current cryotherapy equipment prohibit its massive scale-up for widespread impact on reducing the burden of cervical cancer.

It would therefore be advantageous to provide a device and method that could safely, effectively, and in a low-cost manner treat, destroy or ablate cervical, precancerous lesions in order to prevent cervical cancer.

### SUMMARY OF THE INVENTION

Recognizing the need in emerging nations for the development of improved cryotherapy techniques, the present invention is generally directed to satisfying this need. The present invention's method of treatment or process for providing cryotherapy ablation utilizes a pressurized tank of a low-temperature liquid as its source for the low-temperature, thermal reservoir that is needed to conduct by this process.

A preferred embodiment of the present invention takes the form of a device, that is adapted to connect to the outlet of a tank that is pressurized and contains liquid carbon dioxide for providing a patient with a cryotherapy process that is directed to the ablation of lesions, includes: (a) a piping assembly having a configuration that includes a distal end, adapted to connect to said pressurized tank outlet, and a proximal end with a nozzle adapted to create a spray of carbon dioxide snow as a result of the freezing of the liquid carbon dioxide upon its exhaustion from the piping assembly, (b) a tubular, snow horn having a configuration that includes a distal end, adapted to connect to the piping assembly proximal end, and a proximal end, a boundary wall extending between the ends and enclosing the snow horn's interior region, and wherein the snow horn distal end includes a snow horn nut and pressure relief means, (c) a tubular applicator having a distal end, adapted to temporarily connect to the snow horn proximal end, and a proximal end and an interior wall extending between these ends and enclosing the applicator's interior region, and wherein this interior wall has an interior diameter that increases in value from the proximal end to the distal end of the applicator, and wherein this interior wall has a plurality of vent holes, (d) an applicator tip having an inner surface and an outer surface that includes a metal portion and a configuration adapted to allow the applicator tip to connect to the applicator's distal end during process of providing cryotherapy ablation, and (e) a push rod having a distal end and a proximal end and a base attached to the push rod distal end and wherein this base includes an anchor.

A first variant of this preferred embodiment further includes a handle having a barrel, a grip, a thumb-operated trigger and a drive system adapted to control the movement of the push rod during the process of providing said cryotherapy ablation.

A second variant of this preferred embodiment further includes a wind knob situated on the handle and adapted to control the movement of the push rod during the process of collecting snow in the tubular applicator.

A third variant of this preferred embodiment is adapted so that the handle barrel has a centerline and the handle grip has a centerline, and these centerlines intersect so that the enclosed angle formed by these intersecting centerlines is in the range of 10-20 degrees.

A fourth variant of this preferred embodiment is adapted so that the amount of increase in the diameter of applicator's interior wall between its ends when divided by the length between these ends is in the range of 0.4 to 1.6 percent.

Another preferred embodiment of the present invention is that in which the configurations of the piping assembly, tubular snow horn, tubular applicator and applicator tip configurations are all further adapted for a cryotherapy process that is directed to the removal of cervical, precancerous lesions for the prevention of cervical cancer.

Thus, there has been summarized above (rather broadly and understanding that there are other preferred embodiments which have not been summarized above) the present invention in order that the detailed description that follows may be better understood and appreciated.

### BRIEF DESCRIPTION OF THE DRAWINGS

FIG. 1(a) is a perspective view of the piping assembly of the present invention.

FIG. 1(b) is a perspective view of the snow horn of the present invention.

FIG. 1(c) is a perspective view of the, assembled and filled with CO₂ snow, applicator, applicator tip, push rod, spring and handle of the present invention.

FIG. 2 is an exploded, perspective view of the elements of the present invention shown in FIG. 1(c).

FIG. 3 is a perspective view of an applicator tip that is suitable for use in the present invention.

FIG. 4 is a perspective view of the anchor that is situated on the distal end of the push rod of the present invention.

FIG. 5 is a side view of a snow horn that is suitable for use in the present invention.

FIG. 6 is a side view of a hollow tubular applicator that is suitable for use in the present invention.

FIG. 7 is a cross-sectional view of the hollow tubular applicator shown in FIG. 6.

FIG. 8 is a right side, perspective views of a handle that is suitable for use in the present invention.

FIG. 9 is a left side, perspective views of a handle that is suitable for use in the present invention.

FIG. 10 is a cross-sectional view of the interior of the handle shown in FIGS. 8-9.

### DESCRIPTION OF THE PREFERRED EMBODIMENT

Before explaining at least one embodiment of the present invention in detail, it is to be understood that the invention is not limited in its application to the details of construction and to the arrangements of the components or elements set forth in the following description or illustrated in the drawings. This invention is capable of being practiced and carried out in various ways. Therefore, it is to be understood that the present invention is not to be limited to the specific embodiment disclosed herein, and that its many variants are intended to be included within the scope of the appended claims. Also, it is to be understood that the phraseology and terminology employed herein are for the purpose of description and should not be regarded as limiting. In the accompanying drawings, like numbers refer to like elements throughout.

A preferred embodiment in accordance with the present invention 1 provides an apparatus or device and method that extracts heat from a warmer medium or material such as human or animal tissue. As shown in this application's figures, the embodiment of the present invention that is shown there utilizes cryotherapy to treat, destroy or ablate a patient's abnormal tissues or lesions, e.g., when the patient is a member of the animal species, which includes female humans for the ablation of cervical, precancerous lesions for the prevention of cervical cancer. The device includes an auxiliary piping assembly 10 that is configured to be connected to the typically horizontally-directed outlet of a pressurized, carbon dioxide tank in the bottom of which resides liquid carbon dioxide, CO₂, or other suitable fluid that freezes at a comparatively low temperature (i.e., the phase diagram for CO₂ reveals that it can, when it is under pressures in the range of approximately 5-100 atmospheres, only pass from a liquid to a solid state when its temperature is also below approximately minus 60 degrees Centigrade—we define herein fluids that can remain in a liquid state at such elevated pressures and when approaching such reduced temperatures as low-temperature liquids which upon freezing yield low-temperature snows).

The purpose of this piping assembly and its configuration is adapted to yield the proper flow conditions for the beginning of the creation of the CO₂ snow or “dry ice” from the distal end of the piping assembly when the pressurized tank of carbon dioxide is allowed to exhaust through the piping assembly at operating pressures in the range of 750-850 psig and the CO₂ freezes as it exits the assembly. See U.S. Pat. No. 6,543,251 and European Patent Number (EP) 1,046,614B1 for background technology related to the creation of CO₂ snow.

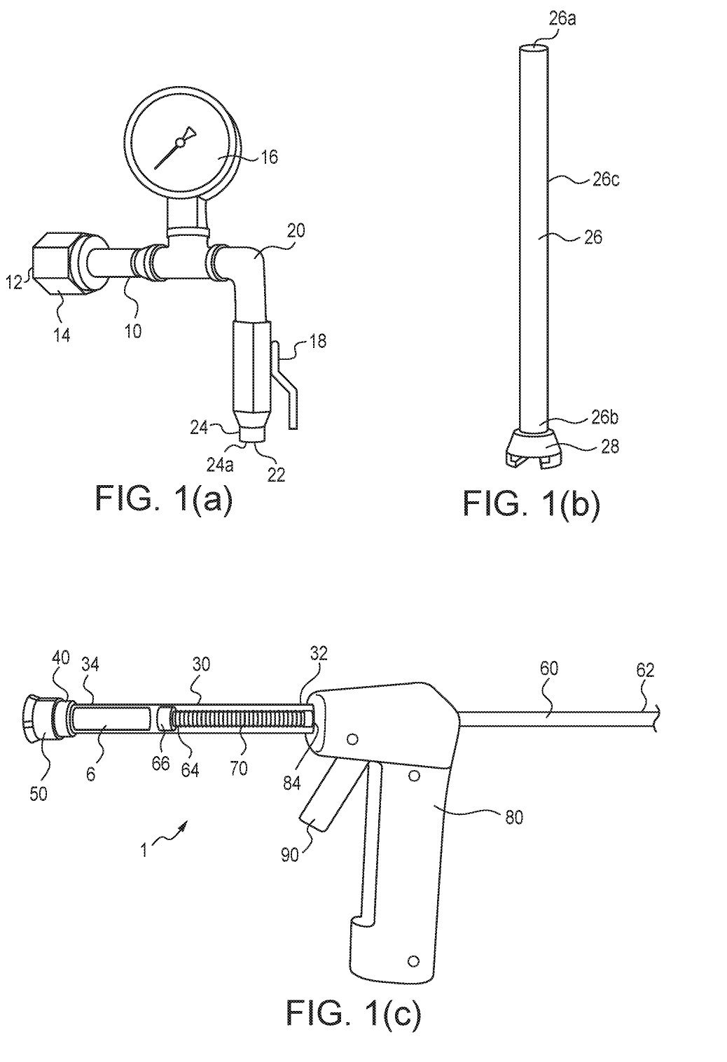

One variant for the configuration of this piping assembly 10 includes a distal end 12 that has an adapter 14 which allows the piping assembly to be connected and firmly affixed to the tank's outlet, while also reducing the diameter of the pipe through which the exhaust is flowing. See FIG. 1(a).

A pressure gauge 16 that is sized to measure and read pressures up to approximately 700-1,000 psig is included in this piping assembly so as to check to ensure that the tank pressure is adequate to support the creation of CO₂ snow. It also includes a valve 18 that regulates flow through the piping assembly. A ninety degree elbow 20 that is used to redirect the normal direction of the flow from the tank from being horizontal to vertical, and a proximal end 22 to which is attached a nozzle 24 whose outlet or orifice 24a diameter is critical to the efficient operation of the piping assembly and it's on-demand, capability to begin the creation of CO₂ snow.

A preferred variant of this piping assembly's distal end has the diameter of the pipe to which the nozzle connects being ⅛ of an inch and the diameter of the nozzle's orifice 24a being in the range of 0.005-0.050 inches, with a preferred value of 0.016 inches. Downstream from this nozzle and its orifice 24a is a snow horn 26 which has a boundary wall 26c and a configuration adapted to create the final flow conditions for the collection of CO₂ snow having a desired density. Alternatively, the snow horn has a configuration adapted to create the proper flow conditions at the snow horn's proximal end so as to enable a prescribed mass of the snow of a desired density to be collected in a specified period of time. See FIG. 1(b). This snow horn has a distal end 26a that has a configuration that allows it to be locked to a comparable fitting (e.g., ⅛ inch NPT tap) on the proximal end 22 of the piping assembly. The proximal end 26b of this snow horn has a special fitting or coupling 28 that allows for it to be easily connected to and disconnected from the distal end 34 of the device's applicator 30. This coupling 28 is also configured as the element of the device which has the least structural strength. The purpose of this is to ensure that the device will break apart at this coupling during a situation in which an accident were to occur and an excess CO₂ pressure were to be applied to the device. Typical snow horn dimensions are an outer diameter of approximately 0.50 inches, a wall thickness of approximately 0.125 inches and lengths in the range of 7-9 inches, with a preferred length of 8 inches.

The CO₂ snow's desired density is chosen so that the mass of CO₂ snow collected is sufficient to provide the needed low temperature reservoir while also allowing the scale of the device to be such that it is appropriate for the desired cryotherapy procedure. Experimentation has shown that a CO₂ snow density in the range of 8-11 g/in³ allows the required mass of snow to be in the range of 8-10 g for a normal preventive, ablation of cervical, precancerous lesions procedure.

The present invention also includes a hollow, tubular applicator 30 which has proximal 32 and distal 34 ends. As its name implies, this part of the present invention is used by a health care provider to help precisely locate the point/s on a patient where the invention's cryotherapy will be applied. This applicator 30 has a configuration that is adapted to collect and temporarily store in its interior 36 and proximate its distal end the mass 6 of CO₂ snow that is required (i.e., sufficient to allow the mass of collected snow to serve as the low temperature thermal reservoir for the device after the applicator's distal end has been disconnected from the snow horn and the applicator's tip attached so the applicator and its tip together can be used to perform a cryotherapy ablation).

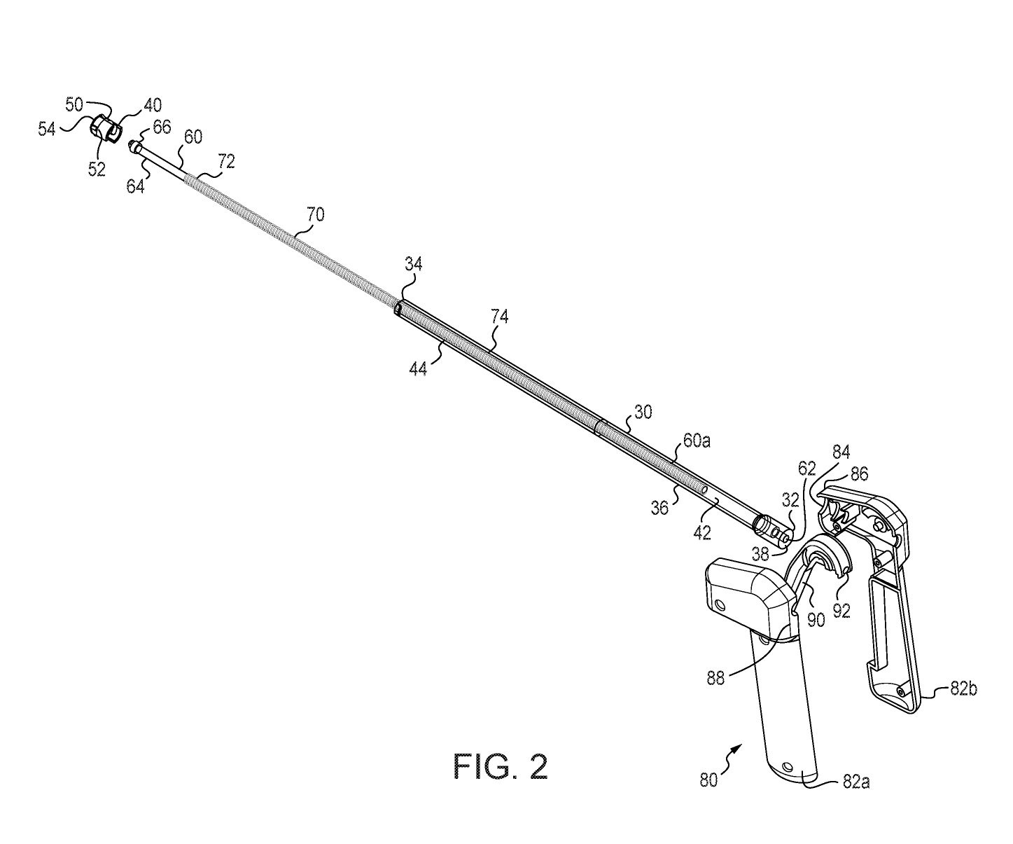

See FIG. 1(c). See also FIG. 2 which is an exploded, perspective view of the elements of the present invention that are shown in FIG. 1(c).

A flow-through fitting 40 is provided at the applicator's distal end that is configured to allow for, among other things, the attachment and detachment of the applicator's distal end to and from the coupling 28 on the distal end of the snow horn during the part of the process in which the applicator is filled with CO₂ snow. This same fitting 40, upon being decoupled from the snow horn, is also configured to allow it to be used for attaching to the applicator's distal end an especially designed applicator cap or tip 50 which is the part of the device that actually comes into contact with a patient's body where it is desired to freeze and ablate targeted tissues or cells.

The proximal end 32 of the applicator has an opening 38 that allows access to the applicator's interior from this proximal end. This opening allows for the introduction of a means for advancing 60 any CO₂ snow that may accumulate in the applicator. For the embodiment shown in FIGS. 1(c) and 2, this means includes a base 66 at the distal 64 end of a retractable, push rod or plunger 60 that can be inserted to different distances into the applicator. The outer boundaries of this base have a configuration that is adapted to allow them to provide a sliding seal against the applicator's interior wall 42. The distal portion of this base, when the push rod is adjustably inserted into the applicator, thus forms the furthest boundary at which CO₂ snow may accumulate in the applicator. The wall 42 of the applicator is also provided with a number of vent holes 44 by which gases can be vented during the CO₂ snow filling process.

In the alternate venting pattern, channels running parallel to the axis of the applicator allow gas venting to the atmosphere at both the proximal 32 and distal 34 ends of the applicator. The opening to each of the channels is sufficiently small such that the particles of snow do not coagulate in this region in such a way as to block the channels. The channels may also serve to vent the sublimating gas during a cryotherapy treatment procedure and thereby prevent accumulation of the sublimate in the vaginal cavity which inhibits the device operator's ability to see the distal end of the applicator.

The exact dimensions of such an applicator will obviously be a function of the type of cryotherapy procedure that is desired to be performed using the present invention. For a normal preventive, ablation of cervical, precancerous lesions procedure, typical dimensions for this applicator are: outer diameter=⅝ inch, wall thickness= 1/16 inch when polycarbonate extruded tubing is being used as the material from which to fabricate the tube, length in the range of 6-8 inches, vent holes: diameters in the range of 0.02-0.03 inches and 15-20 in number and spaced at 0.25 inch intervals along the length of the tube. For an applicator sized in this manner, typical snow horn dimensions would then be: length=7-9 inches, inside tube diameter=⅜ inch and where it may be fabricated from an assortment of plastics, including polycarbonate, acrylic, polysulfone, ABS, acetal copolymer, or polypropelene.

Once the applicator is sufficiently filled with CO₂ snow, the applicator is disconnected from the snow horn and an applicator cap or tip 50 is attached to the fitting 40 on the applicator's distal end 34. During a cryotherapy treatment process, the mass or plug 6 of CO₂ snow must be continually pressed up against the inner side 52 of the applicator cap to ensure that the plug has good thermal contact with the applicator cap 50. An appropriately sized spring or other suitable biasing means 70 is fitted around the push rod and travels along with the push rod and up into the applicator's interior. The distal end 72 of this spring presses against the back side of the base of the applicator while its proximal end 74 rest against a portion of an especially configured handle 80 that has a configuration adapted to allow the handle to be hand-operated to control the positioning of the applicator's tip 50 and either lock in position or incrementally advance the push rod and the plug of CO₂ snow.

Since, during a cryotherapy procedure, the plug 6 of CO₂ snow is slowly moving towards the distal end of the applicator, the interior wall 42 of the applicator near its distal end has been provided with a slight outward taper in order to aid the forward movement of this plug. For a normal preventive, ablation of cervical, precancerous lesions procedure, and consistent with the typical dimensions previously given for an applicator, a typical taper is in the range of 0.5-6.0 percent, or more preferably in the range of 1-3 percent.

The handle 80 of the present invention is of a two-piece 82a, 82b construction and contains an opening 84 in the front face 86 of the handle and into which the proximal end 32 of the applicator is press fitted. This opening extends all the way through the handle and therefore allows the proximal end 62 of the push rod to extend from the rear face 88 of the handle. A spring-loaded trigger 90 is affixed to the handle and is configured such that a boundary edge 92 of the trigger interacts with grooves 60a on the push rod to provide the handle with its ability to incrementally advance or lock the push rod's position relative to the handle. The retraction of the push rod is handled manually (for example, to allow for the filling of the distal end of the applicator with CO₂ snow), i.e., the handle's trigger is pulled backward to release the push rod while the proximal end of the push rod is grasped in the free hand of the one who is holding the device's handle and pulled backward to retract the push rod.

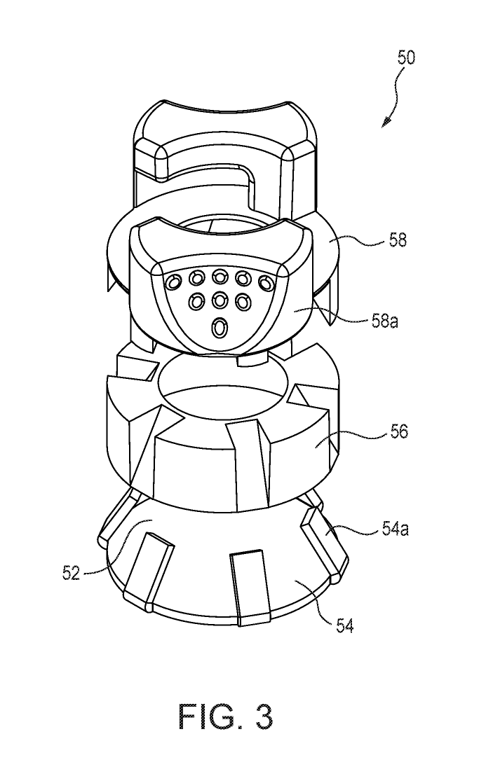

This device's applicator cap or tip 50 has a configuration adapted to allow it to be attached to and detached from the fitting 40 applicator's distal end and provide good thermal transfer through it since it is this tip that comes into contact with a patient and therefore needs an adequate rate of low-temperature thermal transfer from the mass of collected snow to the patient for the cryotherapy ablation process to be successful. This capability is provided by forming its outer surface 54 which comes into contact with a patient from metal while the remainder of the tip is normally formed from plastic. FIG. 3 shows an exploded perspective view of such an applicator tip where an intermediate ring 56 is used to help attaching the taps 54a of the metal outer surface to the ring 56 and it to the tip's connector 58 which has protrusions 58a on either side of it that aid in gripping the applicator tip. The intermediate ring 56 is then attached, usually via gluing, to the tip's connector 58.

The method or steps for using the present invention include:
1. Attaching the piping assembly to a siphoned CO₂ tank such that it's distal end points downward (note: if using a non-siphoned CO₂ tank, the distal end should point upward when the tank is valve side up),
2. Opening the outside packaging of the high level disinfected and/or sterilized components (i.e., snow horn, applicator, push rod, applicator cap, spring, handle) of the present invention without touching the interior of the wrapping.
3. Wiping down a small work surface on which to place the components that are to assembled to make the handle with a solution of 70% or 99% isopropyl alcohol
4. Wiping down the assembled handle and the on/off lever of the piping assembly with a solution of 70 or 90% isopropyl alcohol,
5. Putting on sterile or disinfected gloves,
6. Removing the snow horn and threading or attaching it's distal end to the proximal end of the piping assembly until they are hand-tight, locked together,
7. Removing an applicator and inserting its proximal end into the opening in the handle's front face,
8. Dropping the spring into the applicator,
9. Placing the push rod into the applicator and spring with its proximal end going into and through the handle (squeeze the trigger to allow the push rod to pass all the way through), pulling back on the rod's proximal end once it is through the handle (rather than pushing from the rod's distal end) to completely retract the push rod and then releasing the trigger (don't release the push rod until trigger is completely locked into the grooves of the push rod),
10. Sliding the assembled handle and applicator into the snow horn, the tabs on the applicator's distal end go into the coupling on the snow horn's proximal end (a click will indicate that it is fully seated),
11. On the piping assembly, turning the on/off valve to on; keeping it on until the CO₂ snow is visible above the fitting on the end of the snow horn—then, turning the valve to off,
12. Disconnecting the assembled handle and applicator from the snow horn by sliding it out the way it went in,
13. Sliding the applicator cap onto the distal end of the applicator and twisting it to lock it in place, the device is now ready to perform a freeze,
14. To freeze, simply squeeze the trigger, there will be a slight popping sound as the spring is released,
15. At the end of the freeze, release the trigger and it will lock the push rod in place; for a more rapid defrost, squeeze the trigger and retract the push rod to pull the CO₂ snow back away from the cap and release the trigger to lock the push rod, and
16. After the defrost, squeeze the trigger to unlock the push rod and pull the applicator out of the handle and drop the push rod, spring, applicator, and its cap into a decontaminating, chlorine solution.

For a cryotherapy process directed to the ablation of cervical, precancerous lesions to prevent cervical cancer, and utilizing what is called a “double freeze procedure,” a second freeze would be completed by completing steps 7 through 16 after a five minute thaw cycle is completed and by using a second applicator set (i.e., push rod, spring, applicator and its cap) that would be provided with the device.

An alternative way to describe the method of the present invention is note that it is a method for providing a patient with cryotherapy ablation in which the steps for performing this method utilizing a connection to the outlet of a pressurized tank of a low-temperature liquid in association with the device as disclosed in the prior paragraphs.

It can be seen that the current invention eliminates many of the previously noted disadvantages (e.g., frequent tip blockages in the very expensive, gold or chrome plated nozzles that do not hold up well to frequently being disinfected; current devices on which these nozzles are used cost around thirteen hundred dollars per device; often considerable difficulty in maintaining such devices; need to transport with these devices fifty pound tanks of carbon dioxide tank; difficulty in scaling up the current process to accommodate large numbers of patients) in traditional cryotherapy processes.

It should be noted that many of the above-noted advancements came about only after extensive trial-and-error efforts that utilized a series of prototypes of the present invention. Each of these prototypes also contained improvements and additional features that were added to overcome some of the unexpected, technical problems that were encountered in the operation of the earlier prototypes.

For a first example, more recently the push rod or plunger (60) of the present invention has had the base (66) on its distal end (64) reconfigured so as to better control the rate at which the applicator cap (50) warms up and detaches from the tissue when the cryotherapy procedure is terminated. In earlier prototypes, the dry ice that was at one time adjacent the push's rod's base would occasionally move forward and maintain contact with the applicator cap (50) even after the push rod itself had been locked or retracted. This prevented the applicator cap from immediately beginning to warm up when the cryotherapy procedure was terminated.

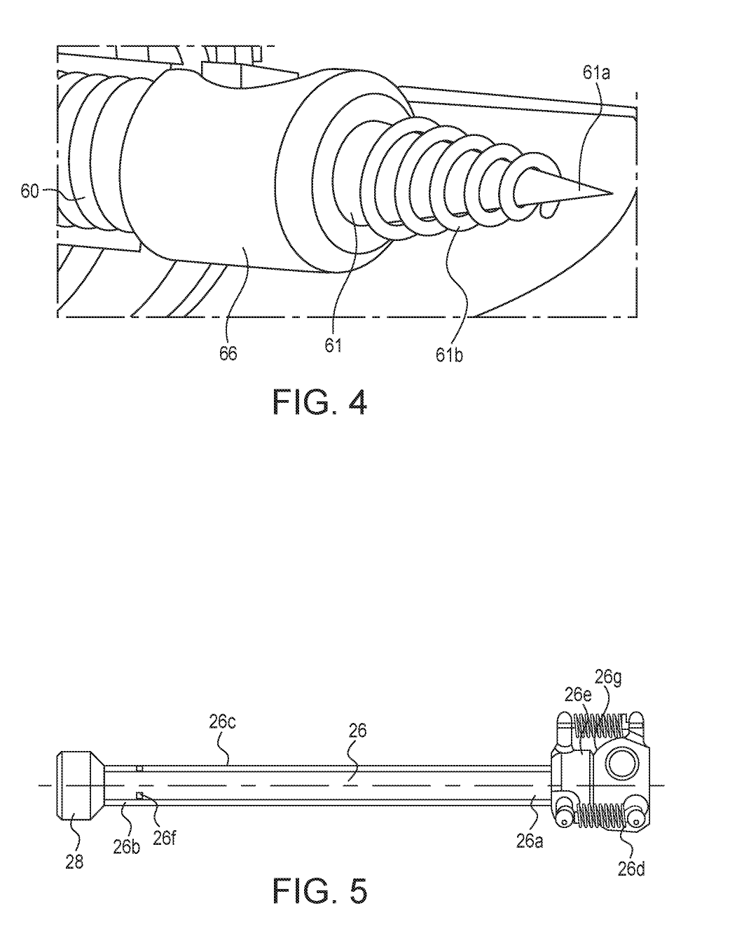

To rectify this situation, a sand-anchor-shaped fitting or anchor 61 was added to the push rod's base. See FIG. 4. This anchor 61 is configured to anchor the low-temperature, CO₂ snow or dry ice to the push rod. This anchor is seen to be cone shaped and to have, on the cone's boundary surface 61a, laterally extending ridges, fins or protrusions or threads 61b which provide more surface area around which the snow can accumulate and that can be used to further adhere the snow to the push rod's base.

For a second example, to make the snow collection process as reliable as possible, it was found desirable to further evolve the present invention's snow horn by improving upon the way that it interfaces at its distal end with the piping assembly proximal end and the way that it vents excess gases during the snow collection process.

FIG. 5 shows a side view of the now modified, snow horn 26 of the present invention. It is seen to have at its distal end 26a a snow horn nut 26d that is adapted to fit onto the piping assembly's proximal end 22.

This nut includes a spring-loaded, pressure-relief collar or other automatic, pressure relief means 26e that is adapted to open at a specified pressure (e.g., approximately 90 psig) to allow excess CO₂ gases to be relieved before the pressure in the snow horn rises to such a level as to threaten the actual rupturing of the snow horn.

Notice should also be taken of the shape of the exterior surfaces of the snow horn nut 26d and the snow horn collar 26e. The have been specifically provided with complemental curvature or a rounded interface 26g at the points where they contact each other so as to enable the snow horn to be pivoted about these contact points to aid in aligning the various components of the device during the snow collection process.

For a third example, to make the tissue cooling portion of a cryotherapy procedure as reliable as possible, it was found desirable to further evolve the present invention's tubular applicator by improving upon: (a) its consistency of performance in allowing the collected quantity of snow to smoothly move forward through the applicator without binding at various points on the applicator's inner wall, and (b) its venting capabilities during the snow collection process.

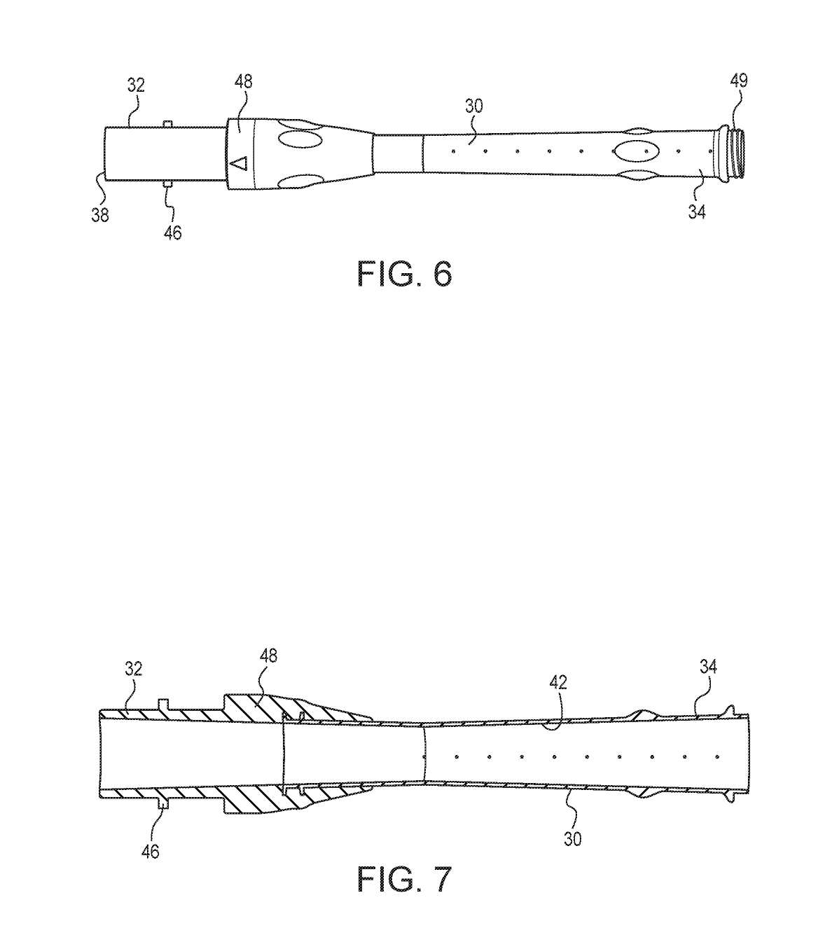

FIGS. 6 and 7 show, respectively, a side view and a cross-sectional view of the now improved-upon, hollow tubular applicator 30 of the present invention. The applicator's interior wall 42 is unique because its diameter is seen to uniformly increase from its proximal to its distal end (i.e., in the direction of advancement of the collected snow during the cooling portion of a cryotherapy procedure). For an applicator that has approximate dimension of length=8 inches and outer diameter=¾ inches, the amount of this increase is on the order 0.07 inches with the interior wall having inside diameters of 0.425 and 0.490 inches at its respective proximal and distal ends. From our experimentation, it appears that, in order to allow a collected quantity of snow to smoothly move forward through the applicator without binding at various points on the applicator's interior wall, the angle of the spread of the interior surface of this wall from its centerline needs to only be in the range of 0.1 to 0.5 degrees when the vent holes themselves have diameters in the range of 0.015-0.030 inches. Alternatively, the amount of increase in the interior wall's diameter between its ends when divided by the length between these ends is in the range of 0.4 to 1.6 percent. Although this taper is comparatively small, after much experimentation with various prototype applicators, tapers of this order of magnitude were found to be essential to allow for the smooth advancement through the applicator of the collected snow without it becoming bound at various points on the applicator's interior.

The quantity and placement of vent holes 44 in the applicator's wall was also, from extensive experimentation, found to be important to the smooth advancement of the collected snow through the applicator. Too many vent holes (e.g, approximately 400 vent holes with 0.02 inch diameters in a 12 inch long, ¾ inch outer diameter applicator) were found to cause too much surface friction and impeded the smooth advancement of the collected snow through the applicator. This decreases the performance of the device by limiting how cold the applicator tip can get.

Venting of the now improved-upon, hollow tubular applicator 30 of the present invention is seen to be accomplished with an assortment of vent holes whose number, locations and diameters are a function of the mass of CO₂ snow or dry ice that is to be collected. For example, for an 8 inch long, ¾ inch outer diameter applicator that's to be used with approximately 12.5 grams of dry ice, such an applicator would typically have: (a) 2 large diameter vent holes that are located near its proximal end and allow CO₂ to vent into the device's handle, and (b) approximately 15-25 vent holes of 0.02 inch diameter that are located around the perimeter of and near the center of the applicator. When only approximately 10 grams of dry ice are to be used with this size applicator, it would typically not have the centrally located vent holes.

This improved-upon tubular applicator 30 is also seen to have: (a) nub locks 46 near its proximal end that aid in locating and locking the applicator into the device's handle 80, (b) coarse threads on its distal end that are used to temporarily connect the applicator to a snow horn during a snow collection process and to the applicator during the freeze process, and (c) a finger grip 48 that aids in aligning the applicator to the device's handle and temporarily locking the components together.

Another embodiment of this improved-upon tubular application includes one that has double walls or tubes (inner and outer) that are separated by an air gap that exists to provide the collected snow within the applicator with insulation from its surrounding hotter environment and to prevent the external surface of the applicator from getting too cold. These tubes are not bonded together so as to allow them to contract at different rates in response to the exposure to extreme temperatures due to contact with dry ice snow.

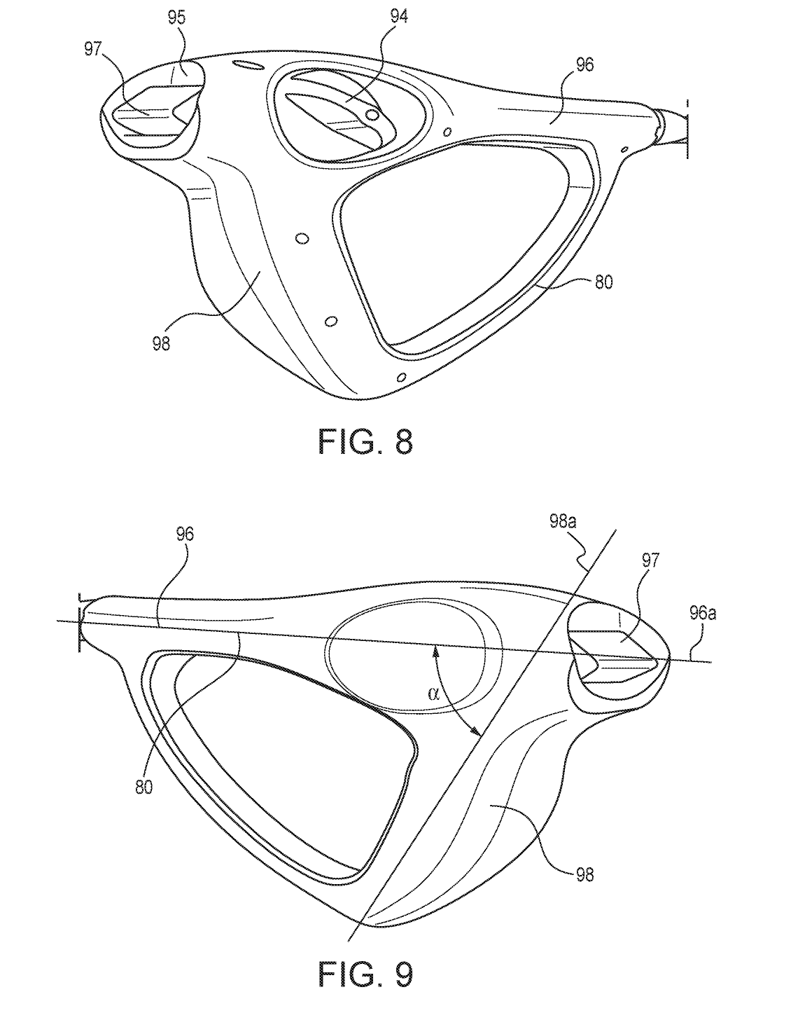

For a fourth example, much experimental effort has been expended towards perfecting the design of the device's handle 80 so as to maximize the reliability of its operation and its ease of use. FIGS. 8-9 show respective right and left side, perspective views of the handle of the present invention. It is seen to have on its right side a wind knob 93 that one uses to wind a cable that is connected to the base 66 of the push rod, and, when this cable is wound onto a drum or spool 94, it causes the push rod to be retracted out of the hollow, tubular applicator 30 into the handle 80 so as to make room in the applicator for the collection of a desired quantity of snow and to compress the coiled spring 99 to create the potential energy that is used to advance the dry ice snalw. At the back, top end of the handle is a concave portion 95 in which sits a thumb-controlled, trigger button 97 that replaces the index-finger activated trigger 90 that was used in the early prototype handles. The center of this trigger button is essentially in line with the centerline 96a or axis of the handle's barrel 96.

Extending downward and forward from the rear portion of the handle's barrel is a grip 98 whose centerline 98a is oriented with respect to the centerline 96a of the handle's barrel so as to be at a specified included angle, α, so as to optimize the ergonomic advantages of the handle. From much experimentation with early handle prototypes, it was found that an included angle in the range of 10-20 degrees was preferred, with an included angle of approximately 12 degrees being optimum.

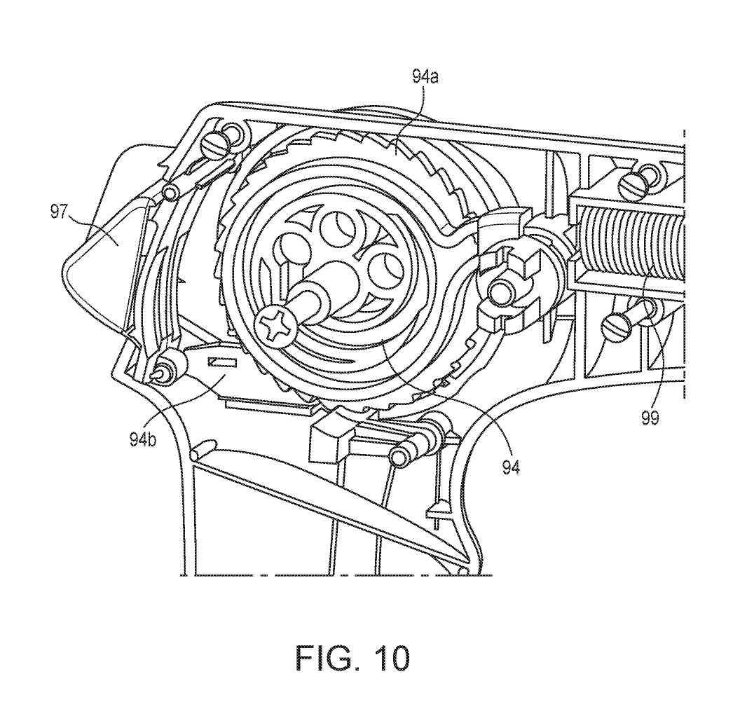

FIG. 10 is a cross-sectional view of the handle's interior and shows the drive and control system for controlling the push rod's movement. The drum 94 onto which the cable is wound contains a toothed, ratchet wheel 94a whose incremental movement occurs when the thumb-controlled, trigger button 97 is pushed downward against the resistance of a torsional spring and causes a link 94b to press against the rachet wheel's paw causing it to disengage from the ratchet wheel's teeth so as to release the potential energy of the spring 99 and allow the base 66 of the push rod to apply the force needed to advance the dry ice snow through the applicator.

The many features and advantages of the present invention are apparent from the above detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to and are considered to fall within the scope of the present invention.

We claim:

1. A device that is adapted to connect to the outlet of a pressurized tank containing liquid carbon dioxide for providing a patient with cryotherapy process that is directed to the ablation of a lesion on said patient, said device comprising:
a piping assembly having a configuration that includes a distal end adapted to connect to said pressurized tank outlet and a proximal end with a nozzle adapted to create a spray of carbon dioxide snow as a result of the freezing of said liquid carbon dioxide upon its exhaustion from said piping assembly,
a tubular, snow horn having a configuration that includes a distal end, adapted to to connect to said piping assembly proximal end, and a proximal end, a boundary wall extending between said ends and enclosing the interior region of said snow horn, and wherein said snow horn distal end having a snow horn nut and pressure relief means,
a tubular applicator having a distal end, adapted to temporarily connect to said snow horn proximal end, and a proximal end and an interior wall enclosing the interior region of said tubular applicator, and wherein said interior wall having an interior diameter that increases in value from the proximal end to the distal end of said applicator, and wherein said interior wall having a plurality of vent holes,
an applicator tip having an inner surface and an outer surface that includes a metal portion and a configuration adapted to allow said applicator tip to connect to said applicator distal end during said cryotherapy process and to allow said metal portion of said outer surface of said applicator tip to come into contact with said patient, and
a push rod having a distal end and a proximal end, and a base attached to said push rod distal end and wherein said base includes an anchor.

2. The device as recited in claim 1, further comprising:
a handle having a barrel, a grip, a thumb-operated trigger and a drive system adapted to control the movement of said push rod during said cryotherapy process.

3. The device as recited in claim 1, further comprising:
a wind knob situated on said handle and adapted to control the movement of said push rod during said process of collecting snow in said tubular applicator.

4. The device as recited in claim 2, wherein:
said handle barrel has a centerline and said handle grip has a centerline and said centerlines intersect so that the enclosed angle formed by said intersecting centerlines is in the range of 10-20 degrees.

5. The device as recited in claim 3, wherein:
said handle barrel has a centerline and said handle grip has a centerline and said centerlines intersect so that the enclosed angle formed by said intersecting centerlines is in the range of 10-20 degrees.

6. The device as recited in claim 1, wherein:
the amount of increase in said interior diameter of said inner tube between said inner tube ends when divided by the length of said inner tube is in the range of 0.4 to 1.6 percent.

7. The device as recited in claim 2, wherein:
the amount of increase in said interior diameter of said inner tube between said inner tube ends when divided by the length of said inner tube is in the range of 0.4 to 1.6 percent.

8. The device as recited in claim 3, wherein:
the amount of increase in said interior diameter of said inner tube between said inner tube ends when divided by the length of said inner tube is in the range of 0.4 to 1.6 percent.

9. The device as recited in claim 4, wherein:
the amount of increase in said interior diameter of said inner tube between said inner tube ends when divided by the length of said inner tube is in the range of 0.4 to 1.6 percent.

10. The device as recited in claim 5, wherein:
the amount of increase in said interior diameter of said inner tube between said inner tube ends when divided by the length of said inner tube is in the range of 0.4 to 1.6 percent.

11. The device as recited in claim 1, wherein:
said tubular snow horn, tubular applicator and applicator tip configurations are all further adapted for when said patient is a human.

12. The device as recited in claim 2, wherein:
said tubular snow horn, tubular applicator and applicator tip configurations are all further adapted for when said patient is a human.

13. The device as recited in claim 3, wherein:
said tubular snow horn, tubular applicator and applicator tip configurations are all further adapted for when said patient is a human.

14. The device as recited in claim 5, wherein:
said tubular snow horn, tubular applicator and applicator tip configurations are all further adapted for when said patient is a human.

15. The device as recited in claim 7, wherein:
said tubular snow horn, tubular applicator and applicator tip configurations are all further adapted for when said patient is a human.

16. The device as recited in claim 1, wherein:
said tubular snow horn, tubular applicator and applicator tip configurations are all further adapted for a cryotherapy process that is directed to the removal of cervical, precancerous lesions for the prevention of cervical cancer.

17. The device as recited in claim 2, wherein:
said tubular snow horn, tubular applicator and applicator tip configurations are all further adapted for a cryotherapy process that is directed to the removal of cervical, precancerous lesions for the prevention of cervical cancer.

18. The device as recited in claim 3, wherein:
said tubular snow horn, tubular applicator and applicator tip configurations are all further adapted for a cryotherapy process that is directed to the removal of cervical, precancerous lesions for the prevention of cervical cancer.

19. The device as recited in claim 5, wherein:
said tubular snow horn, tubular applicator and applicator tip configurations are all further adapted for a cryotherapy process that is directed to the removal of cervical, precancerous lesions for the prevention of cervical cancer.

20. The device as recited in claim 7, wherein:
said tubular snow horn, tubular applicator and applicator tip configurations are all further adapted for a cryotherapy process that is directed to the removal of cervical, precancerous lesions for the prevention of cervical cancer.
